(19) United States
(12) Patent Application Publication
(10) Pub. No.: US 2006/0122781 A1
(43) Pub. Date: Jun. 8, 2006

(54) Method and arrangement for detecting and measuring the phase of periodical biosignals

(76) Inventors: Gunter Henning, Ilmenau (DE); Peter Husar, Ilmenau (DE); Alfred Pecher, Stadt-Lauringen (DE)

Correspondence Address: MAYER & WILLIAMS PC, 251 NORTH AVENUE WEST, 2ND FLOOR, WESTFIELD, NJ, 07090, US

(21) Appl. No.: 10/536,554

(22) PCT Filed: Nov. 27, 2003
(86) PCT No.: PCT/EP03/13355

§ 371 Date: Jan. 9, 2006

(30) Foreign Application Priority Data
Nov. 28, 2002 (DE) 102 55 593.1

(51) Int. Cl. G01N 33/48

(52) U.S. Cl. 702/19

(57) ABSTRACT

The invention relates to a method and an arrangement to detect and measure the phase of periodic bio-signals. The aim of the invention is to detect and measure a causal phase response in periodic bio-signals with improved reliability and higher speed compared to conventional methods, simultaneously reducing the computing power required. According to the invention, a status observer is set up in parallel with the analyzed biological system. The output variables of the biological system and the observer are evaluated in order to minimize the occurring error. The determined phase of a periodic bio-signal can, for example, be used for functional diagnostic purposes.

The invention relates to a method and an arrangement to reliably detect and measure in real time the phase of periodic physiological values or bio-signals.

Methods are known according to the state of the art that use a staggered or sequentially-positioned analysis window across the temporal progression of the bio-signal to determine its phase. Methods or descriptive statistics based on Fourier transforms are applied to the signal section within the window. Thus, for example, periodically illuminating light marks of defined intensity and adequately-high frequency (above about 4 Hz) are used in order to monitor the functional capability of the visual system. An electro-encephalogram (EEG) is compiled for the function test, and the response to stimulus is analyzed with regard to the amplitudes and phase. The phase of the response to stimulus is one of the decisive diagnostic parameters in the functional diagnostic.

A disadvantage of the conventional method is that the statistical unreliability of detection, or the inaccuracy of the measurement, is very high. This uncertainty and inaccuracy result from the signal theory as a result of, and in connection with, the length of the analysis window. The theory states that, as the length of the analysis window decreases, the statistical unreliability and thereby the inaccuracy increases, which is adequately known and has been adequately proved in practical signal analysis. The window must be enlarged in order to achieve a statistically improved result. It is known in the realm of physiology that the phase may alter relatively rapidly, and these alterations are also diagnostically relevant. With lengthier analyse windows the statistical unreliability of the measured result does not show the change and valuable information about phase changes is lost.

It is the task of the invention to provide a method and an arrangement with which it is possible to detect and to measure the causal phase response in periodic bio-signals with better reliability and greater speed, and with simultaneous reduction in computing power, than when using conventional methods.

This task is solved by the invention in that periodic bio-signals are determined corresponding to their physical and physiological source, in that a status observer is set up in parallel with the biological system under analysis, and in that a Kalman filter is used to evaluate the output values of the biological system and of the observer, and to determine the phase.

In the method based on the invention, the phase of a periodic bio-signal is determined and used for functional diagnostic purposes. Thus, for example, a lengthened phase with respect to a healthy test subject may be an important clue to functional problems of the biological system being investigated.

In the arrangement based on the invention, a status observer is arranged in parallel with the biological system being investigated that is imitated by a status model that, corresponding to system model, estimates the status value of phase based on a Kalman filter.

Of advantage here are the facts that the estimation of the phase may occur continuously, and that no staggered or sequentially-applied analysis window is required. This makes analysis of the temporal phase alterations possible. In contrast to a relatively complicated theoretical background of this phase estimator, the practical implementation is simple. In comparison to the conventional method, it requires significantly reduced computing power, so that real-time phase estimation is possible.

In the following, the invention will be described in greater detail using the theoretical derivation and an embodiment example. The pertinent Illustrations show:

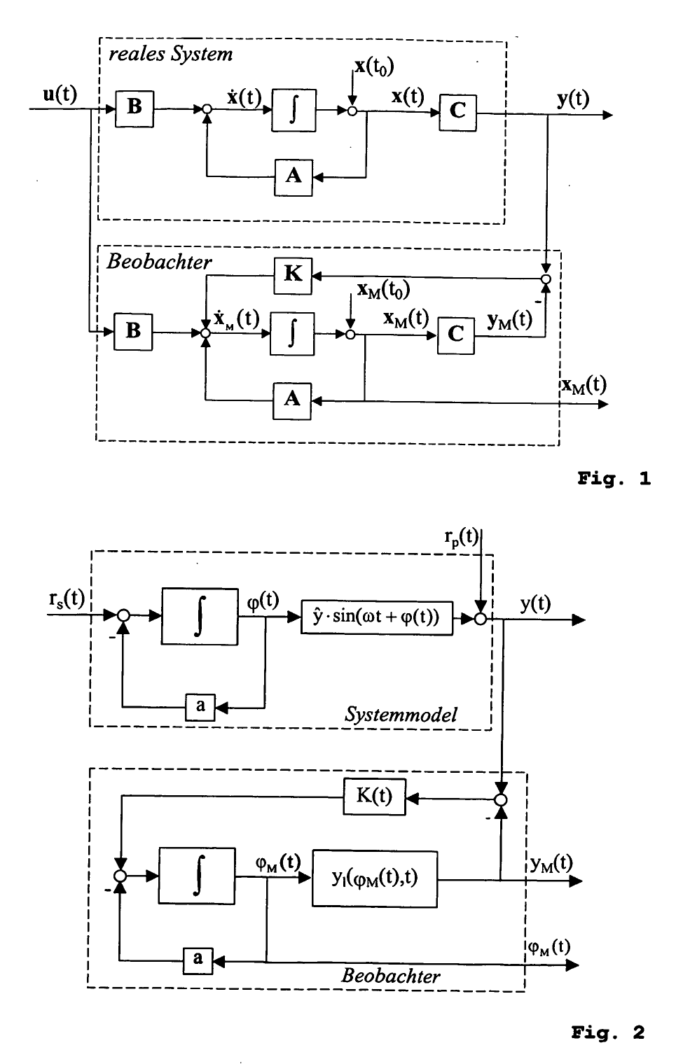

FIG. 1 a flow chart of an observer concept;

FIG. 2 a system model of an arrangement based on the invention;

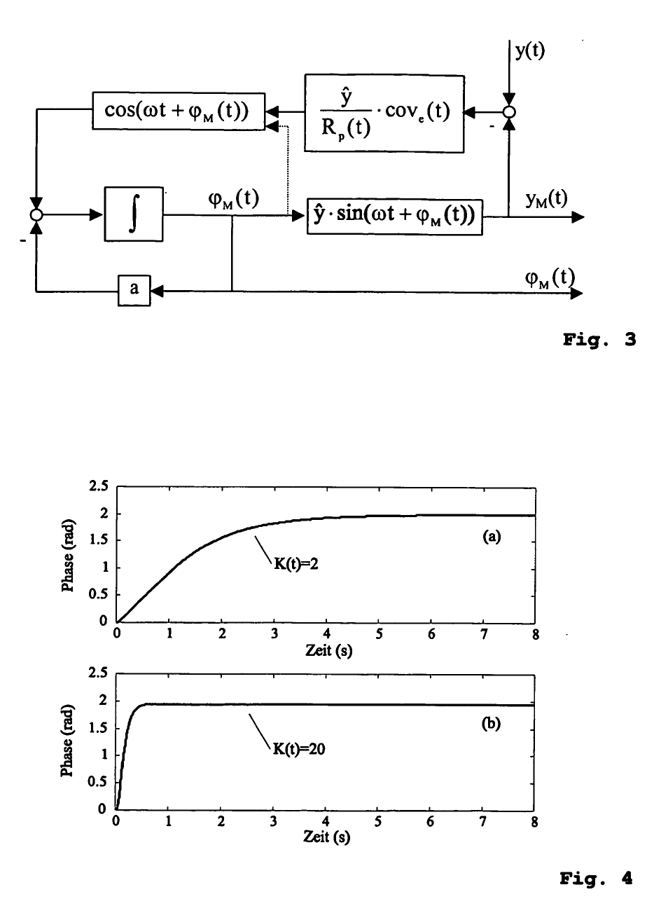

FIG. 3 a representation of the principle of the status observer to measure the phase in periodic bio-signals;

FIG. 4 a progression of an estimated phase for a harmonic of the frequency of 8 Hz and Phase of 2 radians for the static Kalman factors 2 and 20;

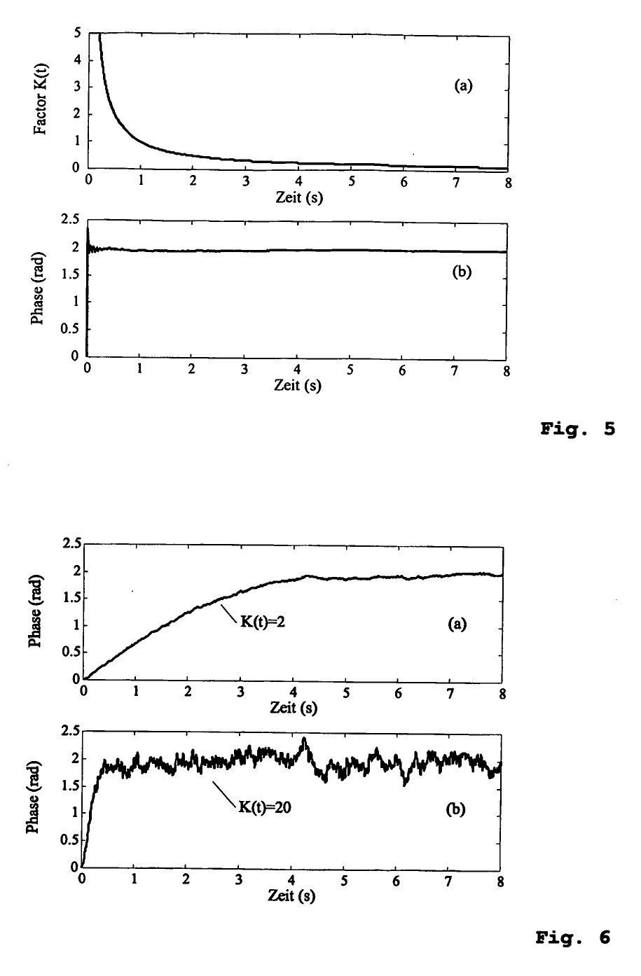

FIG. 5 a progression of an estimated phase for a noise-affected harmonic of the frequency of 8 Hz and Phase of 2 radians with a SNR (signal-to-noise ratio) of 0 dB (lower) and the dynamic Kalman factor (upper);

FIG. 6 estimation of the signal's phase as in FIG. 5 with static Kalman factors;

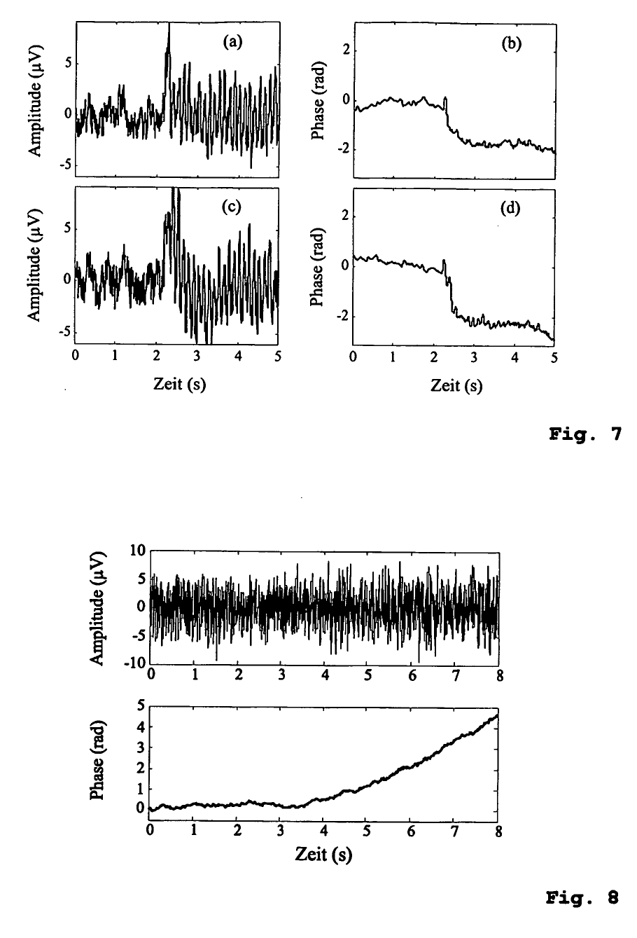

FIG. 7 results of the estimation of the phase (right) of real signals (left);

FIG. 8 an additive overlay of a harmonic of a frequency of 8 Hz with noise and intended de-tuning of the analysis frequency (above) and of the phase progression with acceleration (below).

A biological system that produces a periodic bio-signal or responds to a periodic input signal is shown in FIG. 1 as a status model of a “real system.” The following status equations (1) and (2) describe this system (emboldened capital letters stand for matrices, and small letters stand for vectors):

[equation (1)]

[equation (2)]

For further considerations, an additive signal model is assumed that sums a harmonic oscillation and normally-distributed noise:

[equation (3)]

The goal is to construct a system model whose variable x(t) represents the phase φ(t) of the signal y(t) to be investigated. The phase cannot be measured directly since it is the argument of a trigonometric function. A supplemental construction is therefore required. One such construction is a status observer that is positioned in parallel with the system being investigated. The observer estimates the status variable by minimizing an error function that compares the outputs of the real system with those of the observer. In this manner, the status variable may be measured directly after successful error minimization.

FIG. 1 shows a flow chart of the observer concept. Since x(t) cannot be measured directly, xₘ(t) is estimated within the observer. The inner loop within the observer minimizes the error from yₘ(t) with respect to y(t) with the help of the correction matrix K. For the observer, the status equations (4) and (5) result:

[equation (4)]

[equation (5)]

From (4) and (5), we have:

[equation (6)]

It is assumed that the systems possess different initial conditions. From this, we have the observation error:

[equation (7)]

This observation error disappears iteratively with the help of the correction matrix K, resulting in,

[equation (8)]

The dynamics and stability of the estimation may be described using the differential equation of the observer error (9):

[equation (9)]

Simplification and additional intermediary steps lead to:

[equation (10)]

Corresponding to the signal model (3), one must assume that the investigated signal is destroyed by noise. A Kalman filter is introduced to reduce the influence of noise. Taking the noise into account, the system is described by the following status equations:

System status [equation (11)]

System output [equation (12)]

Observer [equation (13)]

whereby

x(t) is the correction matrix that is to achieve the fact that e(t)=x(t)−x_{M}(t)→0,

e(t)=is the observer error,

rₛ(t) is system noise, and

rₚ(t) is process noise.

In order to simplify the derivation, it is assumed that the noise components are wide-band Gaussian [Nullmittel¹] processes with known co-variances:
¹Translator's Note: Cannot find this term; may mean ‘zero-median.’

[equation (14)]

the noise components are independent of one another, so that:

[equation (15)].

For a constant estimation of x(t), the error performance must be minimized using the matrix K(t):

[equation (16)]

Taking into account the stochastic relationships regarding the co-variances, a suitable correction matrix K(t) is derived corresponding to the Kalman filter:

[equation (17)]

The formula for the error covariance cov_{ε}(t) may be derived from the Kalman filter:

[equation (18)]

Estimation of phase:

The investigated signal is modeled based on (3) from the sum of a harmonic and the noise:

[equation (19)]

The phase results from the differential equation:

[equation (20)]

the system model shown in FIG. 2 results from this. The phase cannot be measured directly. There is therefore parallel to the system an observer in which direct access to the estimated phase φ_{M}(t) is possible. However, the non-linear component yₙₗ(t) in (19) is unfavorable to the observer concept. A suitable linearization yl(φ(t), t) is correspondingly required (5) in order create a linear relationship between the status variables φ_{M}(t) and the output y_{M}(t). Based on the Taylor linearization, the observer may be formulated as follows:

[equation (21)]

[equation (22)]

The observer is modeled based on (21) and (22), as FIG. 2 shows. Equation (22) is linked with Equation (5) in the result of the linearization at the working point φₐ. The factor C that is used in (17) results from this in order to determine the correction factor K(t):

[equation (23)]

The phase to be determined is selected as the working point:

[equation (24)]

and the resulting differential equation for the phase is

[equation (25)].

In accordance with (25), the phase estimator may be modeled, as FIG. 3 shows.

In order to estimate the phase y(t), the error covariance must be calculated. From (18) results:

[equation (26)]

Equation (26) produces a simple solution if higher-frequency components are not taken into account in the error covariance. Based on (27),

[equation (27)],

equation 26 may be simplified to:

[equation (28)].

Upon suitable selection of the parameter a in (28), high-frequency components are suppressed as the result of temporary integration, i.e., it possesses the properties of a low-pass [filter]. Taking the low-pass into account, Equation (25) may be simplified:

[equation (29)].

Thus, the observer (shown in FIG. 3) may be simplified. The system proposed in Equation (29) may particularly be used to estimate phase in harmonics within noise.

FIG. 4 shows the progression of the estimated phase for a harmonic at frequency 8 Hz and phase 2 radians for different Kalman factors. The Kalman factors are static, and equal either 2 or 20. As may be taken from the graph, the estimation becomes slower as the Kalman factor is lower. Static Kalman factors must be used at the point where the point of the phase alteration in time is not known.

FIG. 5 shows the progression (lower part) of the estimated phase for a noise-influence harmonic of the frequency 8 Hz and phase 2 radians with a SNR of 0 dB and dynamic Kalman factor (upper part). If the point in time of the phase alteration is known, then the Kalman factor may be so constructed that resultantly the alteration, and subsequently the variance of the estimation, may be reduced.

In comparison, FIG. 6 shows the phase estimation of the signal itself (in FIG. 5) with static Kalman factors.

FIG. 7 shows the result of a phase estimation of real signals (right column of the graph). Sequences of light pulses with a repeat rate of 8 pulses per second are used to stimulate a visual system under investigation, for example, whereby the light stimulation is followed alternating with a rest pause (time of from 0 to 2 of the time progressions of the signals, upper left and lower left). The progression is shown of an EEG (electro-encephalogram, lift column of the graph) taken from two occipital positions after 16× stimulus-related averaging. Both time progressions show a clear jump in phase after the inception of light stimulation.

Phase estimation becomes problematic for signals strongly influenced by noise. In general, it is true that the phase is more robust against interference than are the amplitudes, as is also known from the realm of Information Technology. However, in this fringe area, the question of the presence (or detection) of a causal phase must be determined, and only then may the phase be estimated.

FIG. 8 shows a harmonic or the frequency 8 Hz being additively overlaid with the noise beginning at t=4 sec, whereby the SNR is −10 dB (upper curve in the graph). In this time interval, the amplitude of the harmonic cannot be established. If one applies the phase estimator with targeted de-tuning, here at a frequency of 7.8 Hz, or 0.2 Hz less than the frequency of the harmonic, then an increase of 1.2 radians/sec in the case of a causal phase (lower curve on the graph). This increase may be used in combination with a discriminator directly to detect the signal.

Reference symbol list

| | |
|---|---|
| a | System parameters, selectable within the observer model |
| A, B, C, K | Matrices in the status model of a system |
| φ(t) | Phase in the system model, value to be estimated |
| φ_{M}(t) | Phase in the observer model, measurable value |
| rₚ(t) | Process noise |
| rₛ(t) | System noise |
| u(t) | Input variable of a system in the status model |
| x(t) | Status variable of a system in the status model |
| x_{M}(t) | Status variable of the observer in the status model |
| y(t) | Output variable of a system in the status model |
| y_{M}(t) | Output variable of the observer in the status model |
| Y₁(φ(t),t) | Linearization operator for phase |

[image]

[image]

[image]

[image]

1. Method to detect and measure the phase of response signals (y(t)) of a bio-system, including the following steps:
a) Multiplication of the response signal (y(t)) whose phase (φ(t)) is to be determined, with a first factor;
b) Multiplication of the product obtained from Step a) by a second factor represented by a trigonometric function, whose argument results from the product of the frequency of the investigated response signal times the time, added to the measured phase, whereby the frequency of the trigonometric analysis function corresponds to the frequency at which the phase is to be determined, or that deviates from this frequency by a known amount,
c) Multiplication of the measured phase times a third factor (a);
d) Formation of a differential from the product obtained in Step b) and the product obtained in Step c);
e) Integration of the differential obtained in Step d) over time, whereby the result of this integration represents the signal's phase to be determined; and
f) Repetition of Steps a) through e) until a break-off criterion is achieved.

2. Method as in claim 1, characterized in that the first factor is chosen to be temporally constant or alterable.

3. Method as in claim 1 or 2, characterized in that, during the differential formation in Step d), the product from Step c) is subtracted from the product from Step b).

4. Method as in one of claims 1 through 3, characterized in that the response signal (y(t)) is directed to a status observer that performs the method steps a) through f) in order to determine an estimated phase (φ_{M}(t)), whereby the method is interrupted when the observer output signal (y_{M}(t)) deviates to be less than an error value (e(t)) specified by an error function (covₑ(t)) from the response signal (y(t), and whereby, after interruption of the method, the estimated phase (φ_{M}(t)) is set to be equal to the phase (φ(t)) of the response signal.

5. Method as in claim 4, characterized in that the Steps a) through d) are performed within the observer according to the following formula:
φ_{M}=−a•φ_{M}(t)+cov_{ε}(t)•y• cos(ωt+φ_{M}(t))•y(t)•R_{F}⁻¹(t).

6. Arrangement to detect and measure the phase of response signals of a bio-system, characterized in that the arrangement includes a status observer in parallel to the bio-system into which the response signal of the investigated system is inserted, and that performs the method steps as in one of claims 1 through 5.

7. Arrangement as in claim 6, characterized in that the status observer includes a Kalman filter with which interfering signals may be filtered out of the response signal.
